(19) United States
(12) Patent Application Publication
(10) Pub. No.: US 2003/0125711 A1
(43) Pub. Date: Jul. 3, 2003

(54) Flexible marker band

(76) Inventors: Tracee E.J. Eidenschink, Wayzata, MN (US); Scott E. Arndt, St. Michael, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 10/263,390

(22) Filed: Oct. 2, 2002

Related U.S. Application Data
(60) Provisional application No. 60/327,232, filed on Oct. 4, 2001.

(51) Int. Cl. A61M 25/098

(52) U.S. Cl. 604/529

(57) ABSTRACT

A catheter having a catheter lumen defined by a catheter wall wherein at least a portion of the catheter wall has at least one radiopaque member. The at least one radiopaque member comprises a body and a weakened portion. The body has a body length, a body thickness and a body circumference. The weakened portion is defined by the body and has a weakened portion length, a weakened portion thickness and a weakened portion width, the weakened portion thickness being less than the body thickness.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Medical devices such as stents, stent-grafts, grafts, or vena cava filters and catheters, balloon catheters, and medical balloons for their delivery are utilized in a number of medical procedures and situations, and as such their structure and function are well known.

Catheters for example, may be used in a variety of medical procedures. An example of one potential use for a catheter is in PTCA procedures. In typical PTCA procedures, a guiding catheter is percutaneously introduced into the cardiovascular system of a patient through a vessel and advanced through therein until the distal end thereof is at a desired location in the vasculature. A guidewire and a dilatation catheter having a balloon on the distal end thereof are introduced through the guiding catheter with the guidewire sliding through the dilatation catheter. The guidewire is first advanced out of the guiding catheter into the patient's coronary vasculature and the dilatation catheter is advanced over the previously advanced guidewire until the dilatation balloon is properly positioned across the lesion. Once in position across the lesion, the flexible, expandable, preformed balloon is inflated to a predetermined size with a liquid or gas at relatively high pressures, to radially compress the arthrosclerotic plaque of the lesion against the inside of the artery wall and thereby dilate the lumen of the artery. The balloon is then deflated to a small profile so that the dilatation catheter may be withdrawn from the patients vasculature and blood flow resumed through the dilated artery.

In angioplasty procedures of the kind described above, there may be injury to or restenosis of the artery, which either necessitates another angioplasty procedure, a surgical by-pass operation, or some method of repairing or strengthening the area. To strengthen the area and help prevent restenosis, a physician can implant an intravascular prosthesis for maintaining vascular patency, commonly called a stent, inside the artery at the lesion. The stent is expanded to a larger diameter for placement in the vasculature, often by the balloon portion of the catheter. Stents delivered to a restricted coronary artery, expanded to a larger diameter by a balloon catheter, and left in place in the artery at the site of a dilated lesion are shown in U.S. Pat. No. 4,740,207 to Kreamer and U.S. Pat. No. 5,007,926 to Derbyshire, the content of which is incorporated herein by reference. Palmaz et al., 156 Radiology 73 (1985) and U.S. Pat. No. 4,733,665 describe introduction of a stent over a balloon catheter (incorporated herein by reference).

To assist in accurate placement of the catheter and stent underneath the lesion site it is useful to visually monitor the catheter as it advances through a vessel. Fluoroscopes or other similar X-ray emitting devices are used to view the catheter within the body as it is advanced. However, in order for the catheter to be visible when exposed to X-rays, the catheter or a portion of the catheter, must be radiopaque to X-rays. In previous catheter designs, radiopaque marker bands, stent retaining members, hubs, catheter tips, or other components have been attached to the catheter for this purpose.

An example of a catheter which utilizes an external metal radiopaque marker band is U.S. Pat. No. 5,759,174 to Fischell et al., the entire contents of which are hereby incorporated by reference, which has a single external metal marker band which is intended to identify the central portion of a stenosis once the delivery catheter is removed. Marker bands such as those disclosed by Fischell et al. are mounted externally on the balloon of an angioplasty catheter and undesirably increase the profile of the catheter, as well as its cost. Furthermore, such marker bands are constructed from expensive and heavy radiopaque metals such as gold, platinum and tantalum or alloys of these dense materials. The use of these heavy materials typically result in marker bands that are somewhat inflexible which may impair the trackability of the delivery catheter.

Despite these shortcomings, marker bands are preferable over radiopaque tips alone, since a radiopaque catheter tip only provides for the end of the catheter to be visible as opposed to a desired area along the catheter shaft. For example, U.S. Pat. No. 5,429,597 to Demello et al., the entire contents of which are hereby incorporated by reference, discloses a balloon catheter having a radiopaque distal tip composed of a polymer mixed with a radiopaque powder such as tungsten.

Other references are known which provide for various radiopaque polymer complexes. For example: U.S. Pat. No. 4,866,132 to Obligin et al.; U.S. Pat. No. 5,256,334 to Smid et al.; and U.S. Pat. No. 5,024,232 to Smid et al., respectively disclose various methods of making radiopaque polymer complexes. In addition, the use of a compliant material to form a marker band which surrounds the external surface of a catheter body is described in U.S. Pat. No. 5,948,489 to Hopkins.

In addition to utilizing radiopaque marker bands for observing the catheter as it is advanced through a body lumen, radiopaque materials may also be utilized in the formation of other potential catheter components such as hubs, bumpers, stops and others.

In delivering stents, it is important to prevent slippage of the stent on the catheter. A number of techniques to prevent slippage have been disclosed. Copending, commonly assigned U.S. application Ser. No. 09/283375 discloses the use of a balloon with protrusions thereon to grip a stent. The use of securement hubs and other stent securement means disposed about the catheter inner tube is disclosed in U.S. Pat. Nos. 6,096,056, 6,007,543, 5,968,069, 5,944,726 and 5,653,691. The use of securement hubs is complicated, however, because when the stent is crimped to the balloon, the force applied to the securement hubs may cause the inner tube to collapse inward. This, in turn, may impair the trackability of the catheter.

In light of the above, it would be desirable to provide for catheter components such as marker bands, hubs, and others components which are engaged to or disposed about the shaft of the catheter and which are further characterized as being radiopaque, flexible and which do not unduly interfere with the trackability of the catheter as it is advanced through the tortuous confines of a body lumen.

The entire content of all of the patents listed within the present patent application are incorporated herein by reference.

### BRIEF SUMMARY OF THE INVENTION

The present invention may be embodied in several forms. In at least one embodiment, the invention is directed to a flexible radiopaque member for use on a catheter shaft wherein the member has one or more weakened portions which act as a hinge or flexing point for the member. The weakened portions of the member are constructed and arranged to allow the member to bend or flex without permanently distorting the shape of the member as the catheter is advanced through a body lumen. The unique flexibility provided to the member allows a catheter utilizing such a member as a marker band to be advanced through a body lumen with out significant compromise to catheter traceability. When such a member is used as a stent retaining hub, the hub provides sufficient rigidity to allow a stent to be crimped thereon without damage to the underlying catheter shaft and also without significant impairment of catheter traceability.

In some embodiments of the invention the members may be a single band of material with one or more, cuts, grooves, perforations, scoring lines or spaces which have been formed on or through the member to form the weakened portions. Such weakened portions may have a variety of configurations. One type of configuration is in the form of a single spiral line or cut extending along the length and about the circumference of the member. Where such a spiral cut extends through the entire thickness of the member and extends along the entire length of the member, the member may be characterized as a ribbon of material.

In some embodiments of the invention, where the weakened portions are spaces or gaps, the band may be characterized as a plurality of individual member segments separated by the spaces.

In some embodiments of the invention the members may be constructed from a variety of radiopaque materials such as bismuth, gold, platinum, tungsten, and/or tantalum in combination with polymer materials such as TECOTHANE, HDPE and/or other polymers.

### BRIEF DESCRIPTION OF THE SEVERAL VIEWS OF THE DRAWINGS

A detailed description of the invention is hereafter described with specific reference being made to the drawings in which:

FIG. 1 is a perspective view of an embodiment of the invention;

FIG. 2 is a side elevational view of an embodiment of the invention;

FIG. 3 is a perspective view of an embodiment of the invention;

FIG. 4 is a side elevational view of an embodiment of the invention;

FIG. 5 is a side elevational view of an embodiment of the invention;

FIG. 6 is a side elevational view of an embodiment of the invention;

FIG. 7 is a side elevational view of an embodiment of the invention;

FIG. 8 is a side elevational view of an embodiment of the invention;

FIG. 9 is a side elevational view of an embodiment of the invention;

FIG. 10 is a side elevational view of an embodiment of the invention; and

FIG. 11 is a graphical comparison of various marker band configurations.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

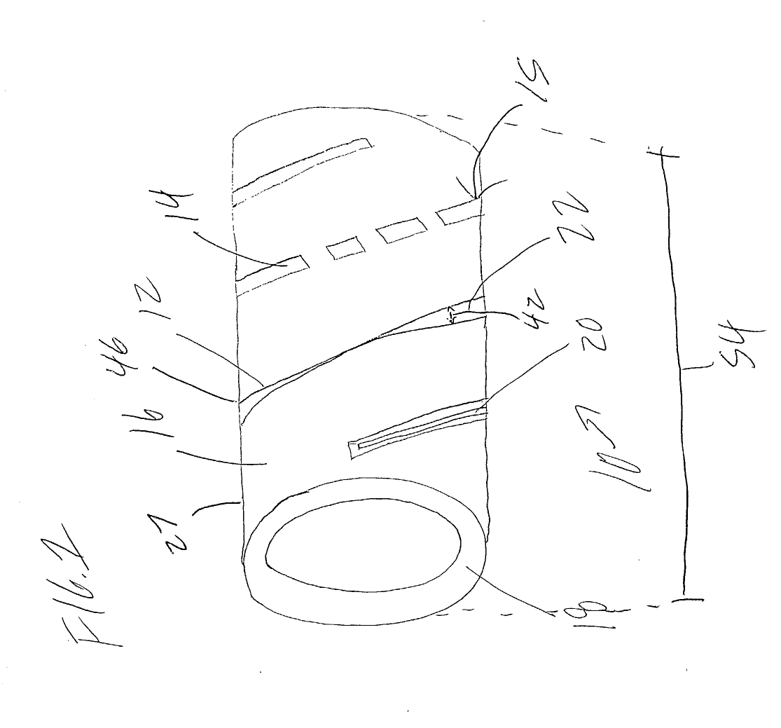

As indicated above, the present invention may be embodied in a variety of forms. In FIG. 1 a first embodiment of the invention is shown wherein a radiopaque marker member, indicated generally at 10, includes at least one weakened area 12 which provides member 10 with improved flexibility. The weakened areas 12 may be a thinner area or area of partial thickness 14 relative to the surrounding material 16 of the member 10. In these embodiments the thinner area 14 may be characterized as a groove or score. Alternatively, the weakened areas 12 may be in the form of cuts 20 which extend through the full thickness 18 of the member 10. Cuts 20 may be incisions which extend through the full thickness 18 to provide improved flexibility of material 16, and which may allow adjacent sections of material to flex, buckle or other wise move relative to one another. Alternatively, the cuts 20 or thinner areas may include gaps or spaces 22 of predetermined width in the member material 16.

As is clear from the above description, FIG. 1 depicts an embodiment of the invention having a variety of weakened area 12 configurations. While such a variety of configurations may be utilized in the member 10, other configurations are possible according to the present invention.

Turning to FIG. 2, an embodiment of the invention is shown wherein the member 10 is depicted as a component of a catheter 24. Catheter 24 includes an inner shaft 26 about a portion of which the member 10 is disposed. Shaft 26 defines a lumen 25. Shaft 26 may include a medical balloon or other expandable inflation device. The shaft 26 may also support an implantable medical device such as a stent. In the embodiment shown, member 10 includes a weakened area 12 which extends about the circumference 27 of the member 10 in a spiral 15. It should be noted that in the embodiment shown, the tubular structure of the member 10 is retained as the weakened area 12 does not extend to the ends 28 and 30 of the member 10. Such a configuration is referred to herein as a closed end configuration.

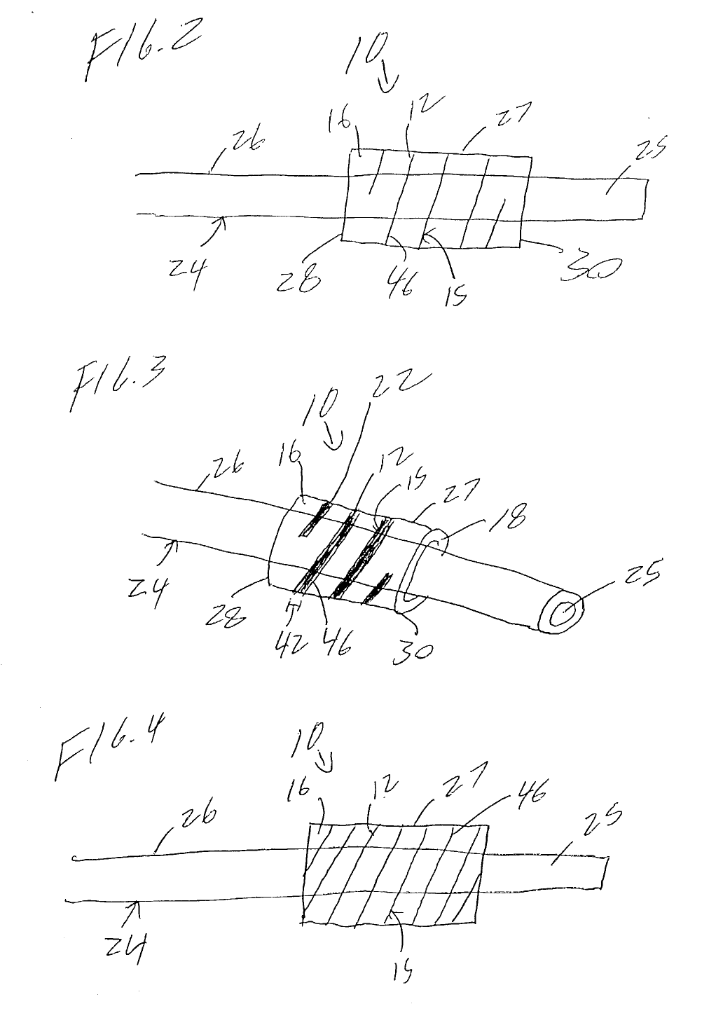

A perspective view of the closed end configuration is shown in FIG. 3.

Where the weakened area 12 extend to the ends 28 and 30 member 10 has what is referred to as an open end configuration, such as is shown in FIG. 4. A member 10 having an open end configuration or closed end configuration may have a weakened portion or portions 12 which extend in the spiral manner shown in FIGS. 2 and 3, or in any other manner suitable to provide the advantageous features of the invention.

Regardless of the particular configuration or manner of design, the weakened portion 12 may be a portion of partial thickness 14 of material 16 or may be a cut 20 extending through the entire thickness 18 of the material 16 or any combination thereof, such as may be seen in FIG. 1.

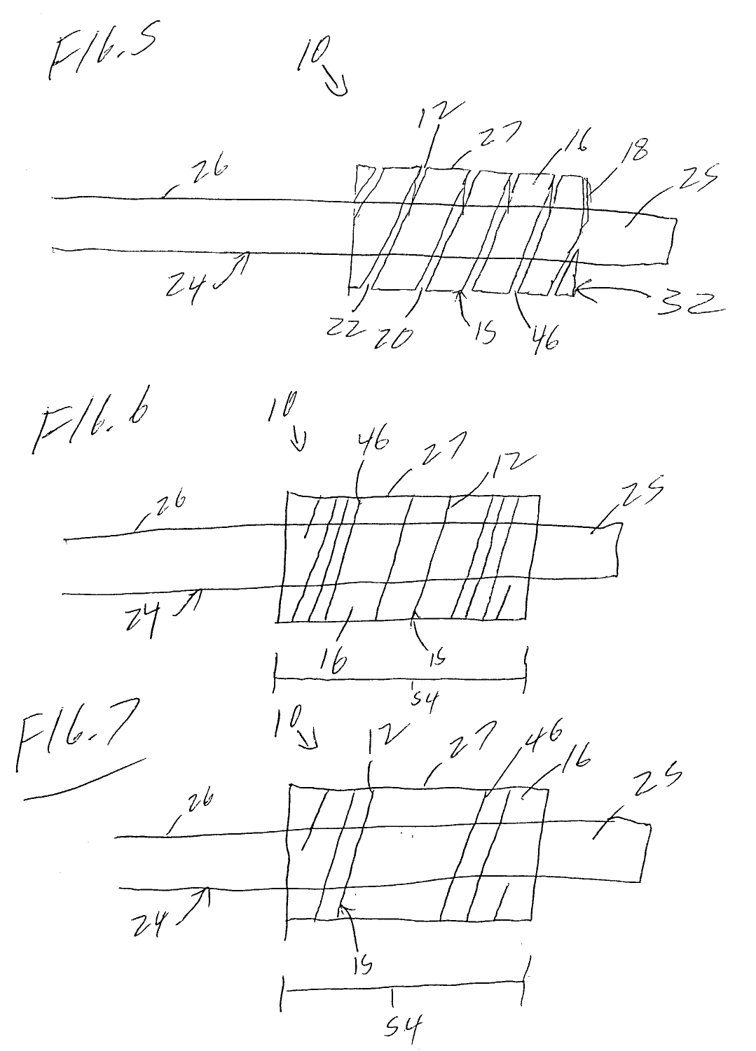

In the embodiment shown in FIGS. 1-4 the weakened portion 12 may define a gap 22 having a predetermined width 42. Both gap 22 and width 42 are shown in FIGS. 1 and 3. Width 42 may be between about zero mm and about 1 mm and may further be in the range of about zero mm to about 0.2 mm. The weakened portion 12 may include gap 22 regardless of whether the weakened portion is a thinner portion 14 or a cut 20 as previously described.

Where the member 10 is an open ended configuration and the weakened portion 12 is a spiral 15 cut 20 extending through the thickness 18 of the member 10, the member 10 may be characterized as a ribbon 32 such as is shown in FIG. 5. The weakened portion 12 of ribbon 32 may also have a gap 22 as is shown.

Ribbon 32 may be a coiled spring. Such a coiled spring may be provided with a closed end configuration such as is shown in FIG. 2, by securing ends 28 and 30 to adjacent material 16.

In those embodiments where the member 10 defines a spiral 15, such as may best be seen in FIG. 1, the spiral 15 may be defined by a number of revolutions or loops 46 about the circumference 27 of the member 10. It should be noted however, that while the various embodiments of member 10 are shown in elliptical or circular profile, the inventive aspects described herein are equally applicable to alternative shapes. While it must initially be noted that the spiral 15 may be defined by any number of loops over a member of a given length, specific numbers of loops may be preferred. In some of the embodiments shown herein, the length 54 of member 10 may vary, between about 0.3 mm or less to about 2.0 mm or less. Preferably, the length 54 of the member 10 is between about 0.7 mm or less to about 1.5 mm or less. Within the length 54 the spiral 15 will define at least 1 loop 46 about the circumference 27 of the member 10. Preferably, the number of loops 46 is between about 2 to about 4.

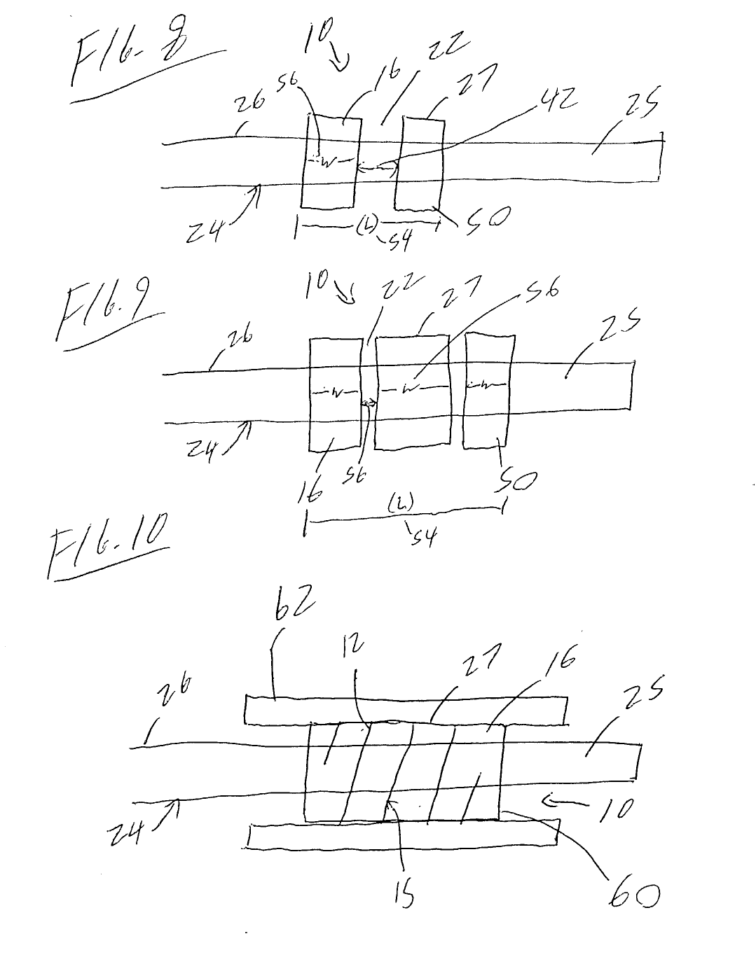

The loops 46 of spiral 15 may be arranged in a variety of manners. For example, in FIGS. 2-5 the turn of the loops are somewhat uniformly spaced along the length 54 of the member 10. In alternative embodiments shown in FIGS. 6 and 7 loops 46 are shown in a variety of different spacing configurations.

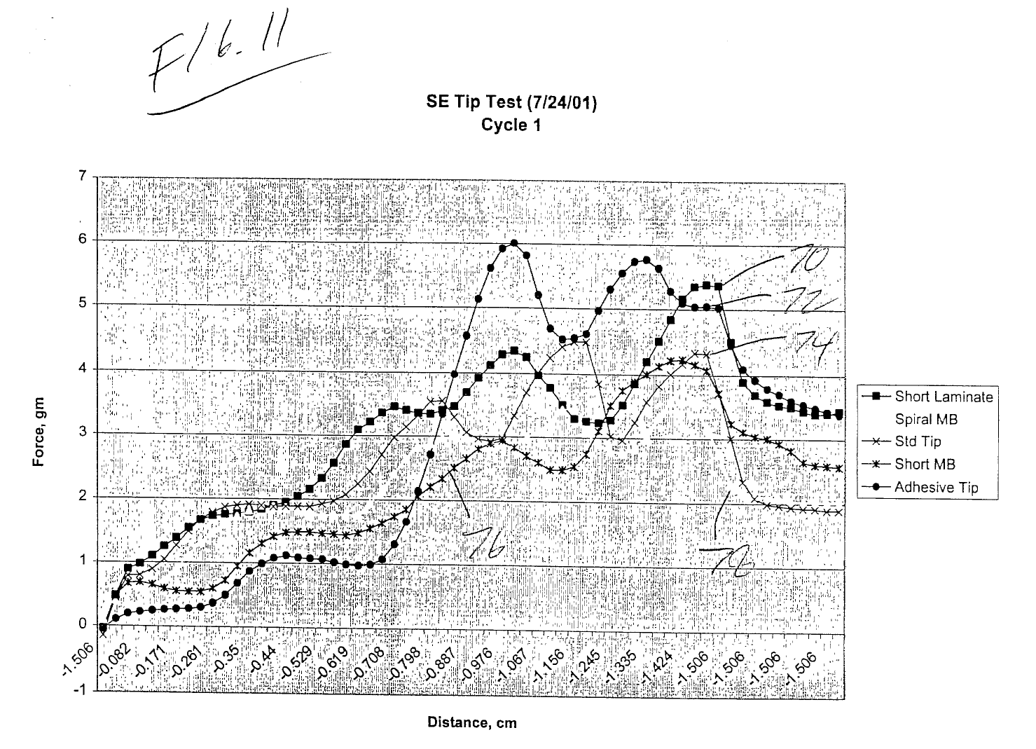

In another embodiment of the invention the member 10 may be comprised of a plurality of member segments 50 separated by gaps 22 such as are shown in FIGS. 8 and 9. Preferably the segments 50 and gaps 22 have a combined length (L) 54 of less than or about 1.5 mm. Each segment preferably has a width (W) 56 of about 0.1 mm to about 0.7 mm, in some other embodiments width 56 may be about 0.3 mm or less to about 0.5 mm or less. Where the member 10 is made up of individual segments 50 the gap 22 between each segment has a width 42 of about 0.5 mm or less.

In the various embodiments shown in FIGS. 2-9, the member 10 may be disposed about the inner shaft 26 of a catheter as a marker band or stent retaining hub. Where the member 10 is utilized as a stent retaining hub 60, such as is shown in FIG. 10, one or more hubs 60 may be used to engage and retain a stent 62 in a reduced configuration prior to stent delivery. Hubs 60 may be integral to the shaft 26 or may be engaged thereto.

In the various embodiments described herein, the weakened portion 12 may be formed by roll cutting with an implement such as a razor blade or other device. Alternatively, weakened portions may be formed by a lathe or through laser ablation or cutting.

It may also be appreciated that member 10 may be formed using known molding and/or extrusion methods.

As mentioned above the members 10 shown and described herein are utilized to provide a catheter or portions thereof with radiopacity so that the advancement and/or position of the catheter may be observed under fluoroscopic or X-ray examination. As a result the material 16 from which the members 10 are made must utilize a radiopaque material in its manufacture. Such radiopaque materials include but are not limited to: bismuth, gold, platinum, tungsten, tantalum, etc. The present also utilizes one or more flexible materials in the manufacture of material 16. Such flexible materials may include but are not limited, polymer materials, silicone, natural rubber, etc. Some examples of some materials 16 may be TECOTHANE™, or HDPE in combination with radiopaque material to provide a desired level of flexibility. Providing a member 10 constructed from such a material 16 with one or more weakened areas 12 improves flexibility even further.

The improved flexibility provided by members 10 is illustrated by the graph shown in FIG. 11. The graph demonstrates the force it took to move a catheter around a 90 degree bend. Lines 70, 72, 74 and 76 depict various catheters having various forms of prior marker bands or tips. Line 78 depicts a catheter equipped with a member 10 on its inner shaft 26 such as is shown in FIG. 2. As may be seen the catheter equipped with member 10 required less force on average to negotiate the turn. This comparison illustrates the improved flexibility of that member 10 provides in practical applications.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1. A flexible radiopaque member for use on the inner shaft of a catheter, the member comprising:
a body, the body having a body length, a body thickness and a body circumference; and
a weakened portion defined by the body, the weakened portion having a weakened portion length, a weakened portion thickness and a weakened portion width, the weakened portion thickness being less than the body thickness.

2. The member of claim 1 wherein the weakened portion further comprises a plurality of weakened portions.

3. The member of claim 1 wherein the weakened portion length is less than the body length.

4. The member of claim 1 wherein the body comprises a first end and a second end, weakened portion length extending from the first end to the second end.

5. The member of claim 1 wherein the weakened portion is selected from at least one member of the group consisting of: groove, indentation, perforation, break, cut and any combination thereof in the body.

6. The member of claim 1 wherein the weakened portion width is less than about 0.5 mm.

7. The member of claim 1 wherein the weakened portion width is less than about 0.2 mm.

8. The member of claim 1 wherein the weakened portion defines a spiral about the circumference of the body.

9. The member of claim 8 wherein the weakened portion length is equal to the body length.

10. The member of claim 9 wherein the weakened portion thickness is zero, the body defining a ribbon.

11. The member of claim 8 wherein the spiral comprises a predetermined number of loops about the circumference of the body, the predetermined number of loops being at least one.

12. The member of claim 8 wherein the loops of the spiral are uniformly disposed about the circumference of the body.

13. The member of claim 8 wherein the loops are arranged in a predetermined pattern.

14. The member of claim 2 wherein the body further comprises a plurality of body segments, each of the plurality of body segments being separated by one of the plurality of weakened portions.

15. The member of claim 14 wherein each of the plurality of body segments has a segment width, each segment width being about 0.5 mm or less.

16. The member of claim 14 wherein each of the plurality of body segments has a segment width, each segment width being less about 0.3 mm or less.

17. The member of claim 14 wherein the weakened portion width is about 0.5 mm or less.

18. The member of claim 14 wherein the weakened portion thickness is zero.

19. The member of claim 1 further comprising a member length, the member length be selected from a range of about 0.7 mm to about 1.5 mm.

20. The member of claim 1 wherein the body is at constructed from a combination of at least one flexible polymer material and at least one member of the group consisting of: bismuth, gold, platinum, tungsten, tantalum and any combination thereof.

21. The member of claim 20 wherein the at least one flexible polymer material is a polyurethane material.

22. The member of claim 1 wherein the member is a marker band.

23. The member of claim 1 wherein the member is a stent retaining hub.

24. A catheter comprising:
a catheter lumen the catheter lumen defined by a catheter wall, at least a portion of the catheter wall having at least one radiopaque member, the at least one radiopaque member having:
a body, the body having a body length, a body thickness and a body circumference; and
a weakened portion defined by the body, the weakened portion having a weakened portion length, a weakened portion thickness and a weakened portion width, the weakened portion thickness being less than the body thickness.

25. The catheter of claim 24 wherein the weakened portion further comprises a plurality of weakened portions.

26. The catheter of claim 24 wherein the weakened portion length is less than the body length.

27. The catheter of claim 24 wherein the body comprises a first end and a second end, weakened portion length extending from the first end to the second end.

28. The catheter of claim 24 wherein the weakened portion is at least one groove, indentation, perforation, break, or cut in the body.

29. The catheter of claim 24 wherein the weakened portion width is less than about 0.5 mm.

30. The catheter of claim 24 wherein the weakened portion width is less than about 0.2 mm.

31. The catheter of claim 24 wherein the weakened portion defines a spiral about the circumference of the body.

32. The catheter of claim 31 wherein the body comprises a first end and a second end, weakened portion length extending from the first end to the second end.

33. The catheter of claim 32 wherein the weakened portion thickness is zero, the body defining a ribbon.

34. The catheter of claim 31 wherein the spiral comprises a predetermined number of loops about the circumference of the body, the predetermined number of loops numbering between one to at least 4.

35. The catheter of claim 31 wherein the loops of the spiral are uniformly disposed about the circumference of the body.

36. The catheter of claim 31 wherein the loops are arranged in a predetermined pattern.

37. The catheter of claim 25 wherein the body further comprises a plurality of body segments, each of the plurality of body segments being separated by one of the plurality of weakened portions.

38. The catheter of claim 37 wherein each of the plurality of body segments has a segment width, each segment width being about 0.5 mm or less.

39. The catheter of claim 37 wherein each of the plurality of body segments has a segment width, each segment width being less about 0.3 mm or less.

40. The catheter of claim 37 wherein the weakened portion width is about 0.5 mm or less.

41. The catheter of claim 37 wherein the weakened portion thickness is zero.

42. The catheter of claim 24 further comprising a member length, the member length be selected from a range of about 0.7 mm to about 1.5 mm.

43. The catheter of claim 24 wherein the body is at constructed from a combination of at least one flexible polymer material and at least one member of the group consisting of: bismuth, gold, platinum, tungsten, tantalum and any combination thereof.

44. The catheter of claim 43 wherein the at least one flexible polymer material is a polyurethane material.

45. The catheter of claim 24 wherein the at least one member is fixedly engaged about the catheter wall.
